(19) United States
(12) Patent Application Publication
(10) Pub. No.: US 2012/0277767 A1
(43) Pub. Date: Nov. 1, 2012

(54) SUTURE PASSER DEVICE AND SUTURE NEEDLE

(75) Inventors: David Powers, Menands, NY (US); Robert G. States, III, Morrow, OH (US); Christoph L. Gillum, Middletown, OH (US); David Parrott, Cincinnati, OH (US)

(73) Assignee: COST CONTAINMENT, INC., Albany, NY (US)

(21) Appl. No.: 13/531,086

(22) Filed: Jun. 22, 2012

Related U.S. Application Data
(63) Continuation of application No. 12/554,703, filed on Sep. 4, 2009, now abandoned.

(51) Int. Cl. A61B 17/04

(52) U.S. Cl. 606/145

(57) ABSTRACT

A suture passer device includes a shaft, a handle, an end effector, and a suture capturing member. The shaft has a shaft proximal end and a shaft distal end. The handle assembly is coupled to the shaft proximal end. The end effector is coupled to the shaft distal end. The suture capturing member is within the end effector, is coupled to the handle assembly independently of the shaft, and is configured to capture a suture within the end effector.

### BACKGROUND

1. Field

The present disclosure relates to a suture passer device and. more particularly, to a suture passer device configured to capture a suture.

2. Description of Related Art

A suture passer device is a tool for passing a suture through tissue, such as through a rotator cuff. Several existing suture passer devices provide mechanisms for capturing the suture after the suture passes through the tissue. However, the existing suture capture mechanisms may not always effectively capture the suture. As such, there is a need for a suture passing device with an improved mechanism for capturing a suture. Furthermore, there is a need for a corresponding suture needle that works in conjunction with the improved suture passing device.

### SUMMARY

In an aspect of the disclosure, a suture passer device includes a shaft, a handle, an end effector, and a suture capturing member. The shaft has a shaft proximal end and a shaft distal end. The handle assembly is coupled to the shaft proximal end. The end effector is coupled to the shaft distal end. The suture capturing member is within the end effector, is coupled to the handle assembly independently of the shaft, and is configured to capture a suture within the end effector.

In an aspect of the disclosure, a suture passer device includes a shaft, a handle assembly, an end effector, and means for capturing a suture. The shaft has a shaft proximal end and a shaft distal end. The handle assembly is coupled to the shaft proximal end. The end effector is coupled to the shaft distal end. The means for capturing a suture is within the end effector and is configured to move responsive to a force applied within the handle assembly.

In an aspect of the disclosure, a suture needle apparatus includes a suture needle, a protrusion, and a circular member. The suture needle includes a suture needle body and a suture needle tip. The protrusion extends from the suture needle body. The circular member is attached to an end of the suture needle body. The circular member is configured to slide within a circular guide.

### BRIEF DESCRIPTION OF THE DRAWINGS

FIG. 1 is a side view of an exemplary suture passer device.

FIG. 2 is another side view of the exemplary suture passer device.

FIG. 3 is an exploded view of the exemplary suture passer device.

FIG. 4a is a view of a suture capturing member within the exemplary suture passer device.

FIG. 4b is a close-up view of a portion of the suture capturing member of FIG. 4a.

FIG. 5 is a close-up exploded view of a portion of the exemplary suture passer device.

FIG. 6 is a cutaway view of the exemplary suture passer device.

FIG. 7 is a perspective cutaway view of the exemplary suture passer device.

FIG. 8 is a close-up cutaway view of a portion of the exemplary suture passer device.

FIG. 9 is a perspective close-up cutaway view of a portion of the exemplary suture passer device.

FIG. 10 is a top view of the exemplary suture passer device.

FIG. 11a is a first perspective view demonstrating capture of a suture with the exemplary suture passer device.

FIG. 11b is a close-up of the end effector of the exemplary suture passer device of FIG. 11a.

FIG. 12a is a second perspective view demonstrating capture of a suture with the exemplary suture passer device.

FIG. 12b is a close-up of the end effector of the exemplary suture passer device of FIG. 12a.

FIG. 13a is a third perspective view demonstrating capture of a suture with the exemplary suture passer device.

FIG. 13b is a close-up of the end effector of the exemplary suture passer device of FIG. 13a.

FIG. 14a is a fourth perspective view demonstrating capture of a suture with the exemplary suture passer device.

FIG. 14b is a close-up of the end effector of the exemplary suture passer device of FIG. 14a.

FIG. 15a is a fifth perspective view demonstrating capture of a suture with the exemplary suture passer device.

FIG. 15b is a close-up of the end effector of the exemplary suture passer device of FIG. 15a.

FIG. 16 is a perspective view of another exemplary suture passer device.

FIG. 17 is a perspective view of the suture needle.

### DETAILED DESCRIPTION

The present invention is described more fully hereinafter with reference to the accompanying drawings, in which various aspects of a suture passer device and a suture needle are shown. This invention, however, may be embodied in many different forms and should not be construed as limited by the various aspects of the suture passer device and the suture needle presented herein. The detailed description of the suture passer device and the suture needle is provided below so that this disclosure will be thorough and complete, and will fully convey the scope of the present invention to those skilled in the art.

The detailed description may include specific details for illustrating various aspects of a suture passer device and a suture needle. However, it will be apparent to those skilled in the art that the invention may be practiced without these specific details. In some instances, well known elements may be omitted to avoid obscuring the inventive concepts presented throughout this disclosure.

Various aspects of a suture passer device and a suture needle may be illustrated with reference to one or more exemplary embodiments. As used herein, the term “exemplary” means “serving as an example, instance, or illustration,” and should not necessarily be construed as preferred or advantageous over other embodiments of the controller disclosed herein. In addition, the term “coupled” means that two elements are connected either directly or indirectly with one or more intervening elements.

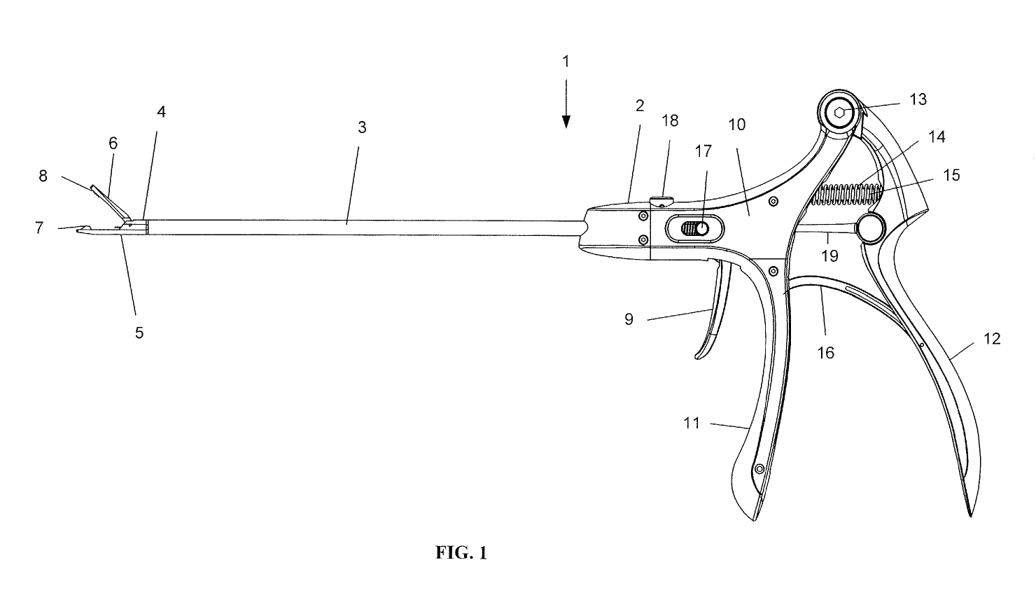

FIG. 1 is a side view of an exemplary suture passer device 1. The suture passer device 1 includes a handle assembly 2, a shaft 3, and an end effector 4. The end effector 4 has a lower jaw 5 and an upper jaw 6. The lower jaw 5 includes ridges 7 between which a suture needle extends and to which a suture needle retracts. The upper jaw 6 may include teeth 8 for gripping tissue/muscle. The shaft 3 may be shaped as a cylinder or a tube. The shaft has a shaft proximal end coupled to the handle assembly 2 and a shaft distal end coupled to the end effector 4. The handle assembly 2 includes a trigger 9 for controlling movement of the upper jaw 6, a front handle assembly 10, and a rear handle 12 connected to the front handle assembly 10. The front handle assembly 10 includes a front handle 11. The rear handle 12 is connected to the front handle assembly 10 at pivot 13. The pivot 13 may be a pin or a bolt. A compression spring 14 is coupled between the front handle assembly 10 and the rear handle 12. The spring 14 may include a guide or bar 15 to keep the spring 14 from buckling. The rear handle 12 may also be connected to the front handle assembly 10 with a handle guide 16 for maintaining the alignment of the rear handle 12. The front handle assembly 10 may additionally include a manual cocking lever 17 and a manual release button 18 for releasing the cocking lever 17. The cocking lever 17 positions a suture capturing member (not shown) that extends from the handle assembly 2, through the shaft 3, and into the end effector 4. The suture passer device 1 is configured to extend a suture needle 19 through the handle assembly 2, the shaft 3, and out the lower jaw 5 of the end effector 4.

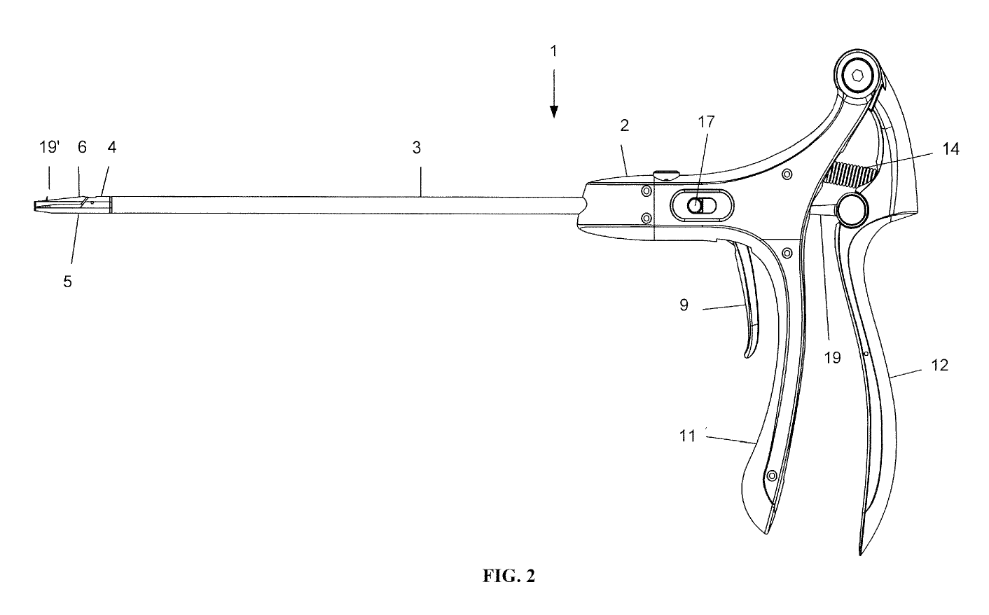

FIG. 2 is another side view of the exemplary suture passer device 1. As shown in FIG. 2, the rear handle 12 is positioned closer to the front handle 11, such that the spring 14 is compressed. In addition, the trigger 9 is pulled back to close the upper jaw 6 onto the lower jaw 5. The rear handle 12 extends the suture needle 19 through the handle assembly 2, the shaft 3, and the end effector 4. The suture needle 19 causes the cocking lever 17 to cock into a cocked position. When the cocking lever 17 is in a cocked position, the suture capturing member (not shown) is positioned to allow a suture and suture needle tip 19′ of the suture needle 19 to extend through the lower and upper jaws 5, 6.

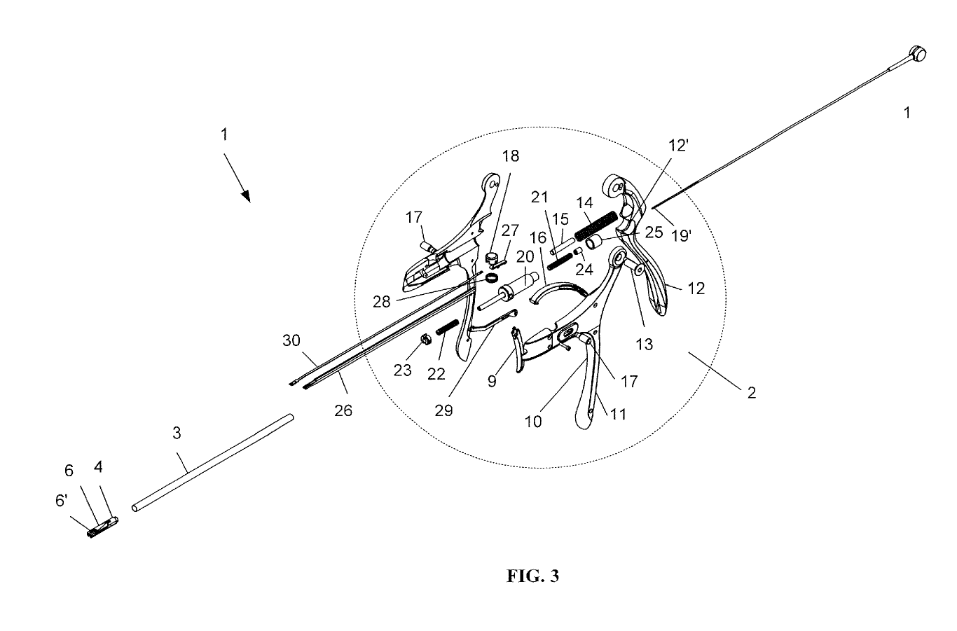

FIG. 3 is an exploded view of the exemplary suture passer device 1. The upper jaw 6 has an opening 6′ through which the suture needle tip 19′ can extend. The handle assembly 2 includes a primary hammer 20, a first compression spring 21 for biasing the primary hammer 20 in a first direction, a second compression spring 22 for biasing the primary hammer 20 in a second direction opposite the first direction, a bar nut 23 coupled to the primary hammer 20, a secondary hammer 24, and a hammer nut 25. The handle assembly 2 also includes a trigger linkage 29 coupled to the trigger 9. The shaft 3 includes a U-shaped coupling bar 26, which is coupled between the upper jaw 6 and the trigger linkage 29. The U-shaped coupling bar 26 and the trigger linkage 29 couple the trigger 9 to the upper jaw 6. The cocking lever 17 and the release button 18 work in conjunction with a latch lever 27 and a compression spring 28. The latch lever 27 latches onto the primary hammer 20 when the cocking lever 17 is in a cocked position. The spring 28 biases the release button 18 up and biases the latching lever 27 down to latch the primary hammer 20. The bar nut 23 is coupled to both the primary hammer 20 and the suture capturing member 30. The suture capturing member 30 may be a bar that extends from the handle assembly 2, through the shaft 3, and into the upper jaw 6 of the end effector 4. Accordingly, the suture capturing member 30 is within the end effector 4 and is coupled to the handle assembly 2 independently of the shaft 3. The suture capturing member 30 is configured to move independently of the end effector 4 longitudinally within the end effector 4. That is, while the suture capturing member 30 is configured to bend with the upper jaw 6 as the upper jaw 6 opens and closes, the suture capturing member 30 is also configured to move longitudinally (i.e., along a length of the upper jaw 6) within the upper jaw 6, and therefore moves independently of the upper jaw 6 when moving longitudinally within the upper jaw 6.

In one configuration, the suture capturing member 30 is nickel titanium (NiTi, also known as Nitinol). The strain on the NiTi suture capturing member 30, due to the movement of the upper jaw 6, is a function of the thickness of the suture capturing member 30. As such, as the thickness of the NiTi suture capturing member 30 is reduced, the strain on the NiTi suture capturing member 30 due to the movement of the upper jaw 6 is also reduced. Accordingly, in one configuration, the NiTi suture capturing member 30 is formed with a thickness such that the strain on the NiTi suture capturing member 30 is lower than an allowable limit for repeated uses (e.g., 50,000 uses).

In one configuration, an exemplary suture passer device includes a shaft, a handle assembly, an end effector, and means for capturing a suture. The shaft has a shaft proximal end and a shaft distal end. The handle assembly is coupled to the shaft proximal end. The end effector is coupled to the shaft distal end. The means for capturing a suture is within the end effector and is configured to move responsive to a force applied within the handle assembly. In one configuration, the means for capturing suture is the suture capturing member 30, which may be a bar that extends from the handle assembly 2, through the shaft 3, and into the upper jaw 6 of the end effector 4.

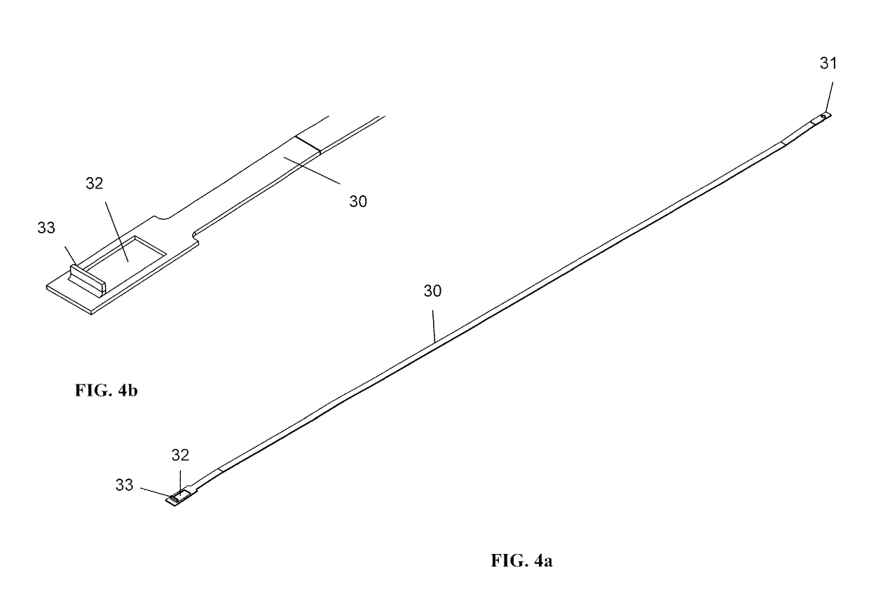

FIG. 4a is a view of the suture capturing member 30. FIG. 4b is a close-up view of a portion of the suture capturing member 30. The suture capturing member 30 has a hole 31 for attaching to the bar nut 23. The suture capturing member 30 also has an opening 32 through which a suture needle and suture may extend. The suture capturing member 30 may include a lip 33 on an edge of the opening 32 so that there is more surface area that can make contact with an inserted suture.

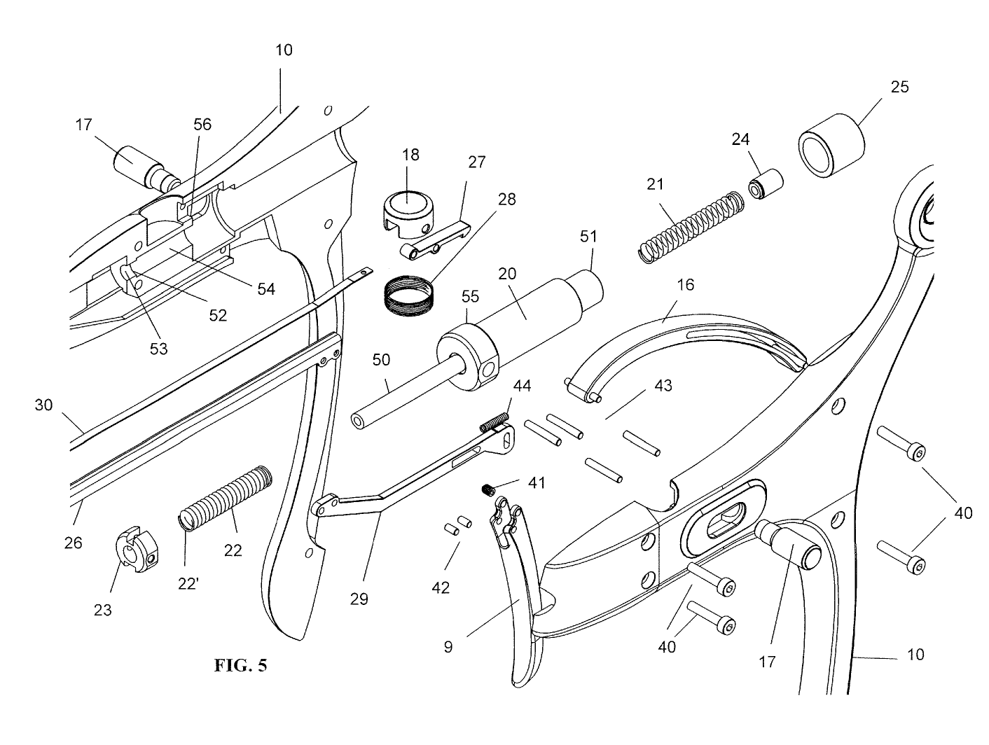

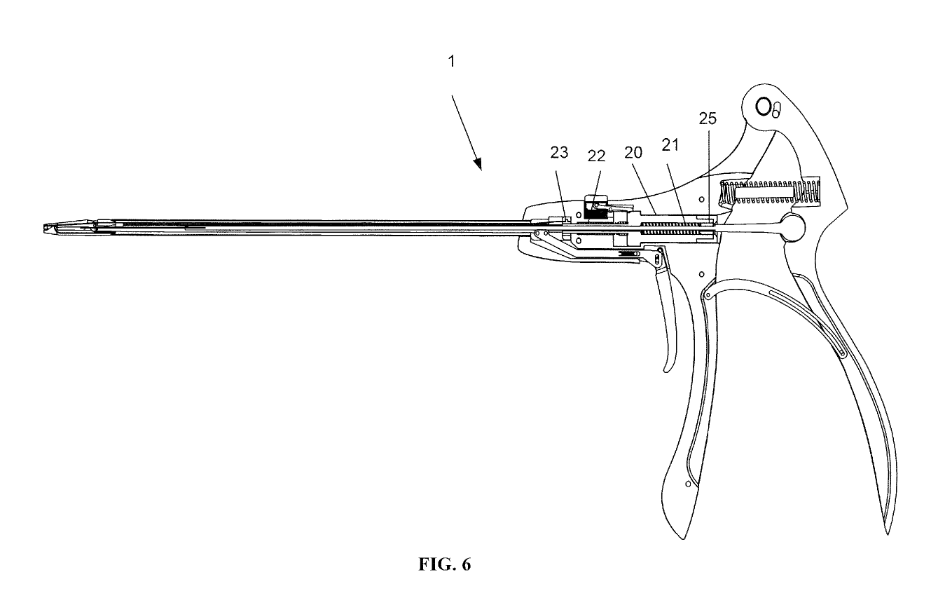

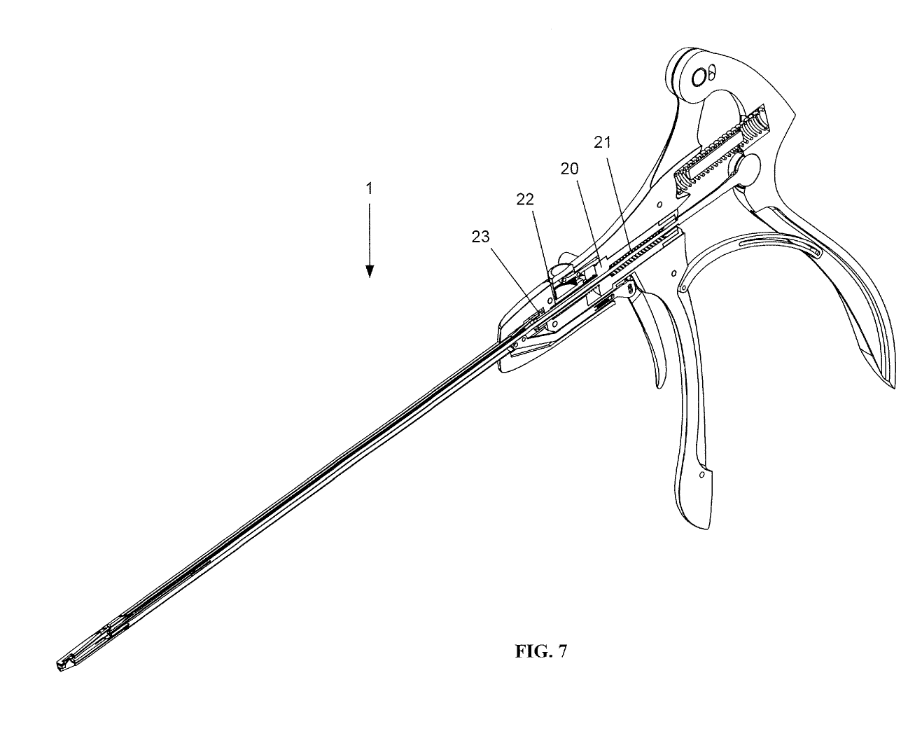

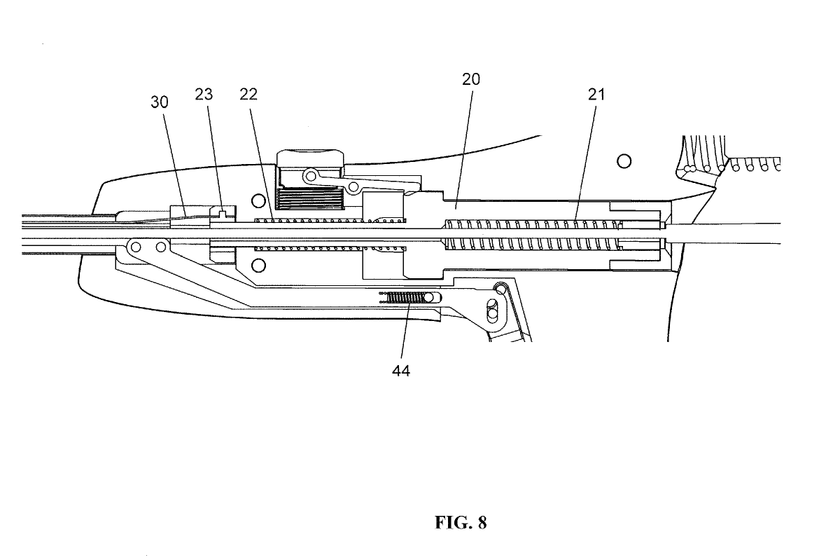

FIG. 5 is a close-up exploded view of a portion of the exemplary suture passer device 1. FIG. 6 is a cutaway view of the exemplary suture passer device 1. FIG. 7 is a perspective cutaway view of the exemplary suture passer device 1. FIG. 8 is a close-up cutaway view of a portion of the exemplary suture passer device 1. The front handle assembly 10 is held together with a plurality of bolts 40. The bar nut 23 is affixed to the primary hammer 20 with a set screw 41. The trigger linkage 29 is coupled to the U-shaped coupling bar 26 with the pins 42. The pins 43 secure the trigger 9 to the trigger linkage 29 and to the front handle assembly 10. The spring 44 biases the trigger 9 such that the upper jaw 6 is biased in an open position. The primary hammer 20 includes a male cylindrical member 50 over which the second spring 22 fits and includes a female cylindrical channel 51 in which the first spring 21 inserts. The secondary hammer 24 is positioned adjacent the first spring 21 within the female cylindrical channel 51 and the hammer nut 25 attaches to the primary hammer 20 to maintain the second hammer 24 within the female cylindrical channel 51 and to preload (i.e., precompress) the first spring 21. An edge 22′ of the second spring 22 rests against a ledge 52 within a cylindrical channel 54 of the front handle assembly 10. The second spring 22 is also preloaded within the cylindrical channel 54. The male cylindrical member 50 inserts into the cylindrical channel 54 through a hole 53 adjacent the ledge 52. The bar nut 23 is attached to the male cylindrical member 50 outside of the cylindrical channel 54.

The first spring 21 and the second spring 22 may be configured with different rates (stiffness). The rate of a spring is the change in the force it exerts, divided by the change in deflection of the spring. The inverse of the spring rate is compliance. In one configuration, the first spring 21 has a higher rate (i.e., is stiffer and has a lower compliance) than the second spring 22.

As a force is applied to the secondary hammer 24 by the needle (which inserts through the hammer nut 25), a force is exerted on the first spring 21. The first spring 21 exerts a force on the primary hammer 20, which exerts a force on the second spring 22. Because the second spring 22 has a higher compliance (i.e., lower rate) than the first spring 21, the second spring 22 compresses more easily than the first spring 21, and therefore before the first spring 21 substantially compresses, the male cylindrical member 50 of the primary hammer 20 slides from a first hammer position within the cylindrical channel 54 until it is stopped in a second hammer position when the circular member 55 of the primary hammer 20 rests against edge 56 of the front handle assembly 10. Upon additional force being applied to the secondary hammer 24, the first spring 21 compresses, as the primary hammer 20 is not able to move any further than the second hammer position. When the primary hammer 20 is in the second hammer position, the suture capturing member 30, which is coupled to the primary hammer 20, is fully extended within the upper jaw 6 of the end effector 4. As the first spring 21 is further compressed, the suture needle tip 19′ extends out from the lower jaw 5, through the opening 32 of the suture capturing member 30 and through the opening 6′ of the upper jaw 6. As the first spring 21 is decompressed, the suture needle tip 19′ retracts back into the lower jaw 5, leaving a suture within the opening 32. Assuming the latching lever 27 is not activated, as the first spring 21 is further decompressed and the second spring 22 is decompressed, the primary hammer 20 moves from the second hammer position to the first hammer position, causing the suture capturing member 30 to withdraw into the upper jaw 6, thus pinching any suture within the opening between lip 33 and an inside edge of the opening 6′ in the upper jaw 6.

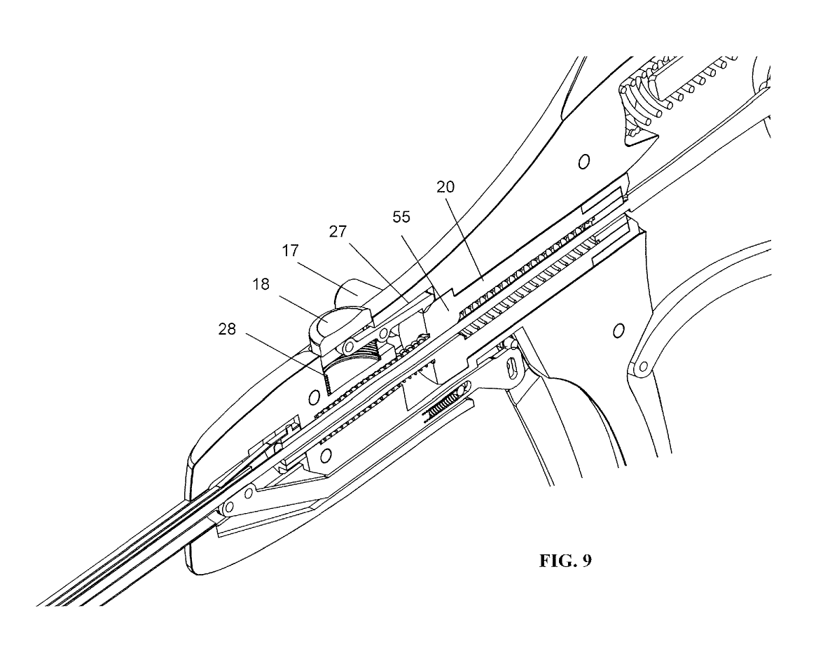

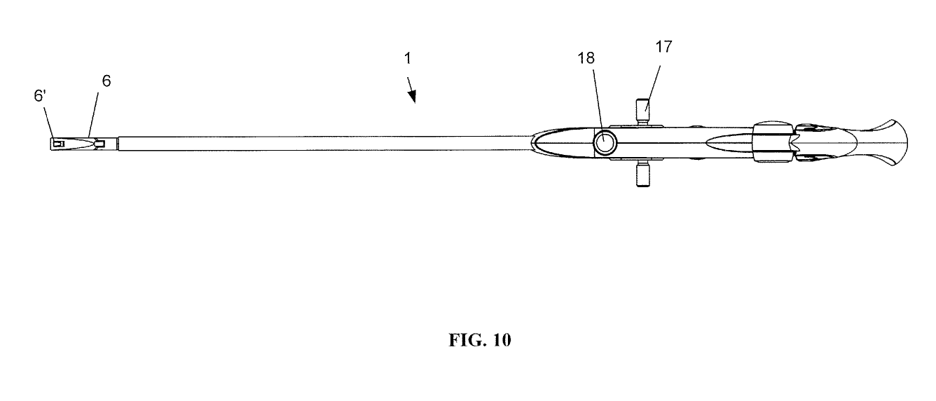

FIG. 9 is a perspective close-up cutaway view of a portion of the exemplary suture passer device 1. FIG. 10 is a top view of the exemplary suture passer device. As shown in FIG. 9 and FIG. 10, the primary hammer 20 is in a first hammer position in which the first and second springs 21, 22 are not substantially compressed. When the primary hammer 20 moves to a second hammer position, the latching lever 27 latches onto an edge of the circular member 55, as the latching lever 27 is biased to latch onto the primary hammer 20 by the spring 28. When the latching lever 27 is latched, the release button 18 extends upward. Once latched, the primary hammer 20 is prevented from moving from the second hammer position. A user may delatch the primary hammer 20 by pressing the release button 18. When the primary hammer 20 is delatched, the primary hammer 20 is forced by the second spring 22 into the first hammer position, thus causing the suture capturing member 30 to snap back to capture any suture within the opening 32 of the suture capturing member 30.

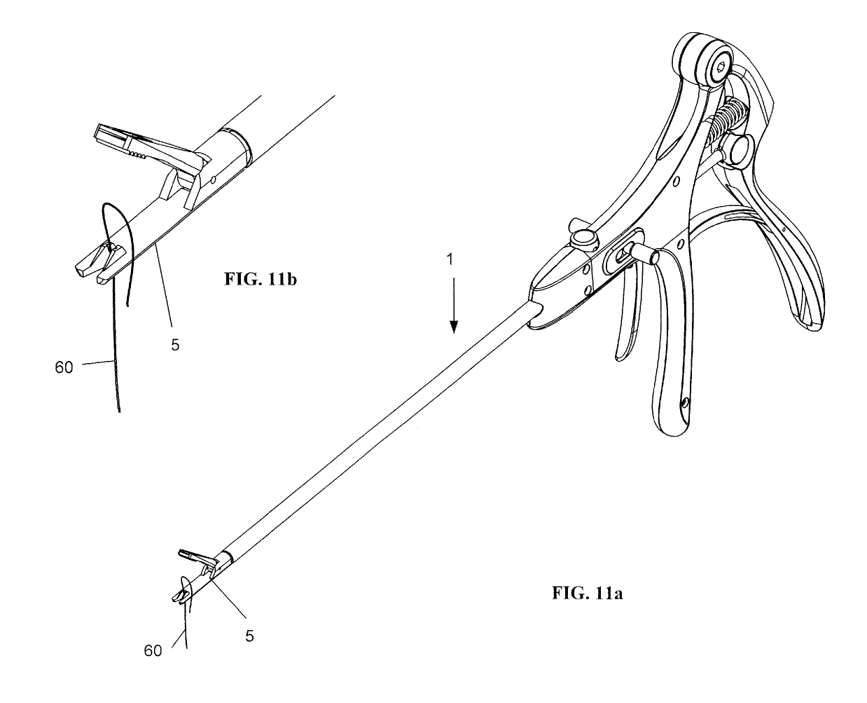

FIG. 11a and FIG. 11b are first perspective views demonstrating capture of a suture 60 with the exemplary suture passer device 1. As shown in FIG. 11a and FIG. 11b, a suture 60 is fed into the lower jaw 5.

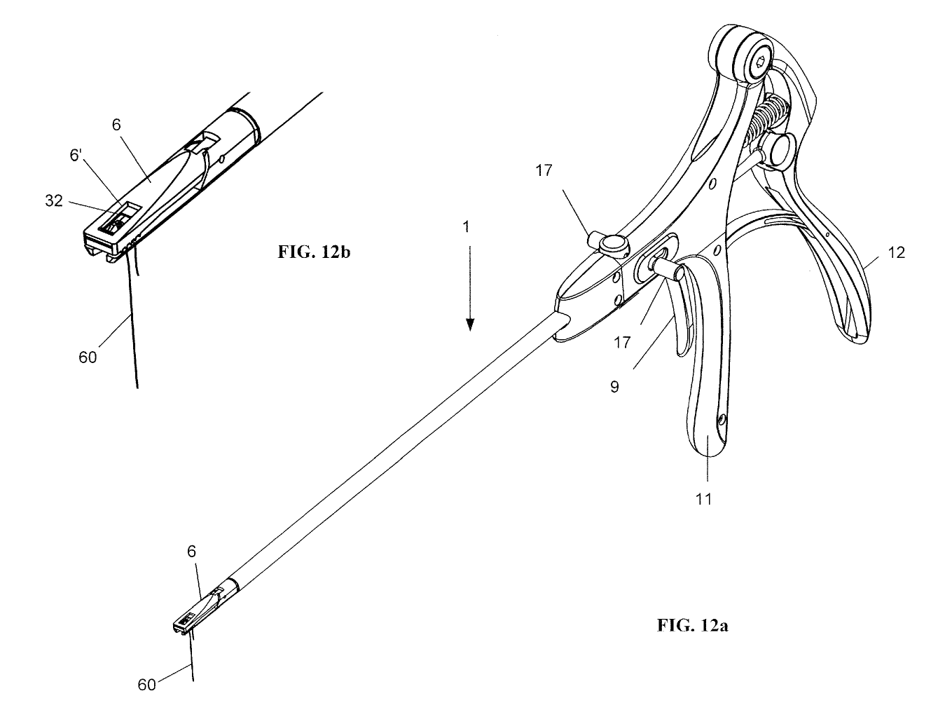

FIG. 12a and FIG. 12b are second perspective views demonstrating capture of a suture 60 with the exemplary suture passer device 1. As shown in FIG. 12a and FIG. 12b, the upper jaw 6 is closed by pulling the trigger 9. In addition, the suture capturing member 30 is fully extended to align the opening 32 with the opening 6′ of the upper jaw 6. In FIG. 12a, the suture capturing member 30 is fully extended by shifting the cocking lever 17 forward. However, as described supra, the suture capturing member 30 may be fully extended by applying a force on the second handle 12 towards the first handle 11.

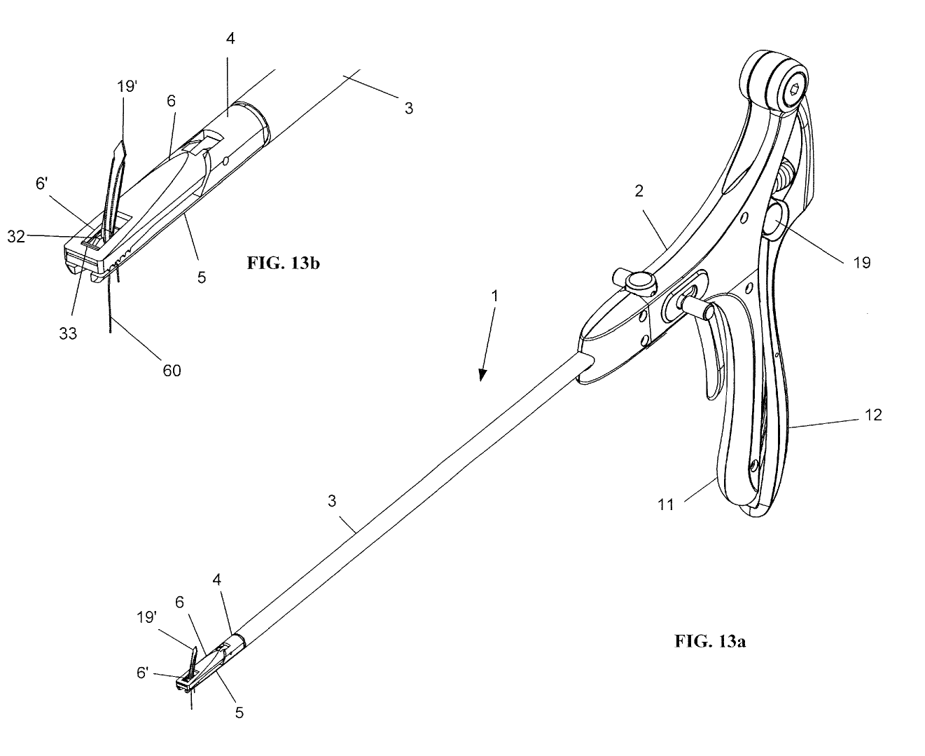

FIG. 13a and FIG. 13b are third perspective views demonstrating capture of a suture 60 with the exemplary suture passer device 1. As shown in FIG. 13a and FIG. 13b, a force is applied on the second handle 12 towards the first handle 11, thus forcing the suture needle 19 to extend through the handle assembly 2 and the shaft 3, and causing the suture needle tip 19′ to extend out of the lower jaw 5, and through the opening 32 of the suture capturing member 30 and the opening 6′ of the upper jaw 6. As the suture needle tip 19′ extends through the openings 32, 6′, the suture needle tip 19′ carries with it the suture 60.

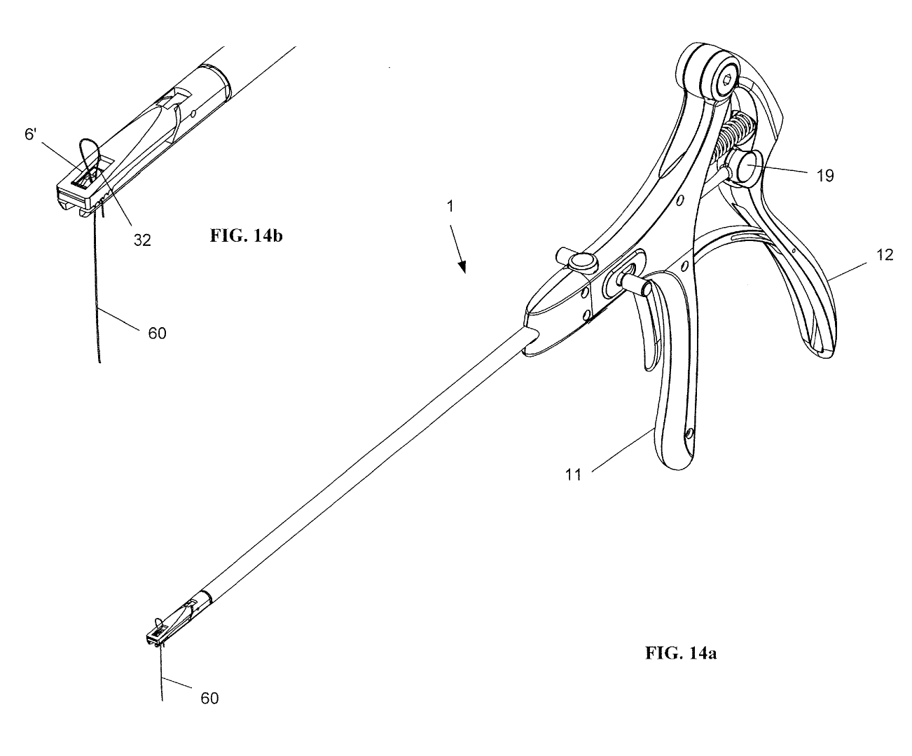

FIG. 14a and FIG. 14b are fourth perspective views demonstrating capture of a suture 60 with the exemplary suture passer device 1. As shown in FIG. 14a and FIG. 14b, the force on the second handle 12 towards the first handle 11 is released, allowing the suture needle 19 to retract within the lower jaw 5, thus leaving the suture 60 within the openings 32, 6′.

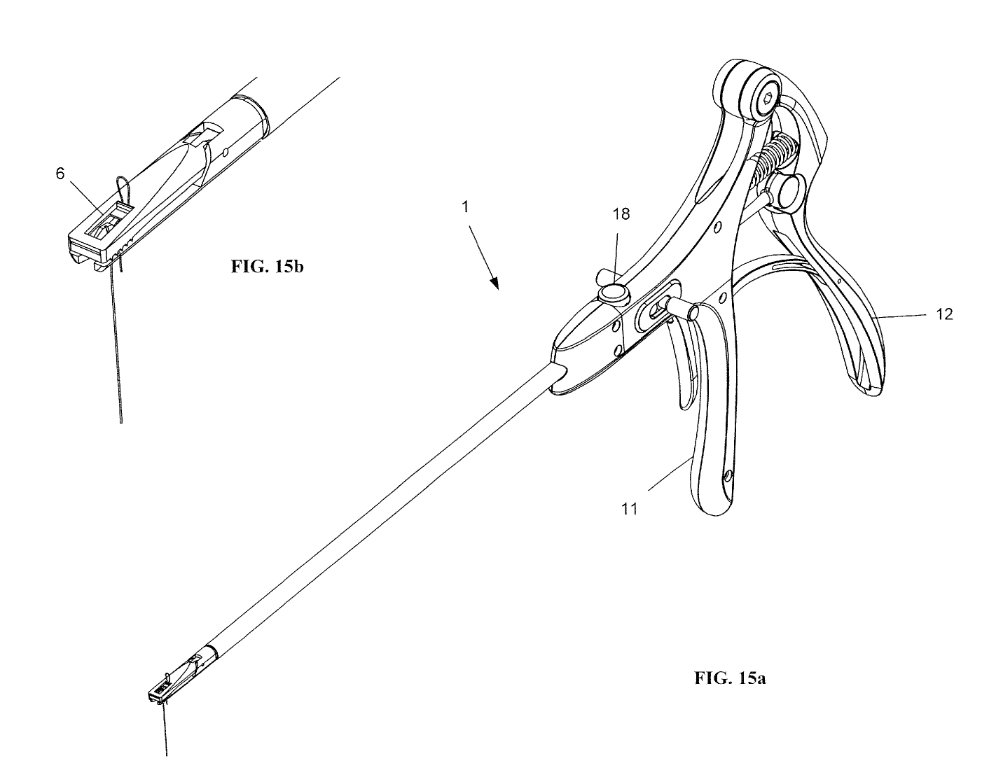

FIG. 15a and FIG. 15b are fifth perspective views demonstrating capture of a suture 60 with the exemplary suture passer device 1. As shown in FIG. 15a and FIG. 15b, the suture 60 may be captured by the suture capturing member 30 by pressing the release button 18, which allows the suture capturing member 30 to retract within the upper jaw 6, thus pinching the suture 60 between inside edges of the openings 32, 6′.

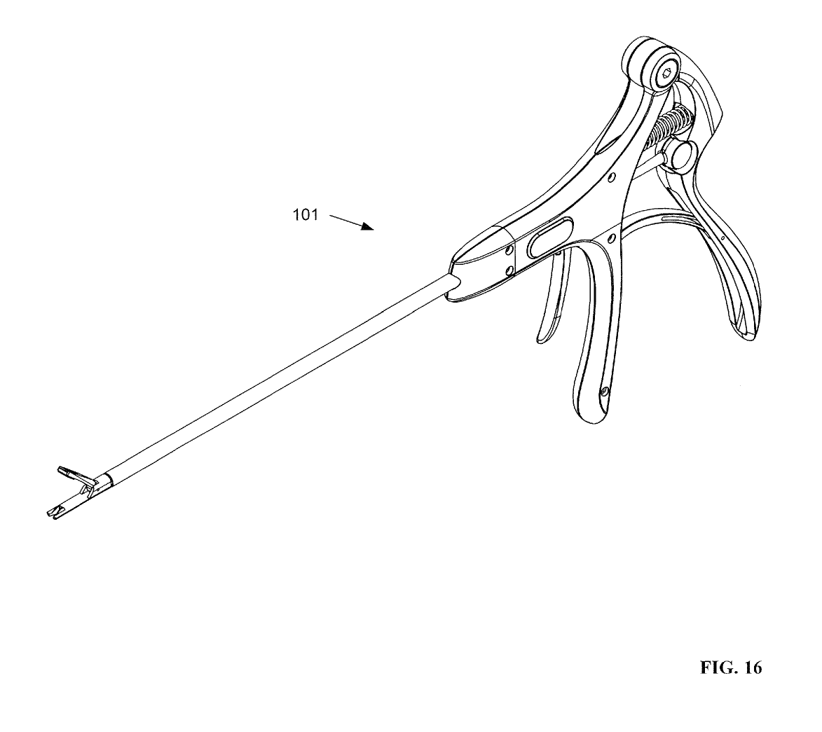

FIG. 16 is a perspective view of another exemplary suture passer device 101. The suture passer device 101 does not include the release button 18, the cocking levers 17, or any mechanisms to lock the primary hammer 20. As such, in this configuration, the suture capturing member 30 automatically captures a suture when force on the rear handle 12 towards the front handle 11 is released.

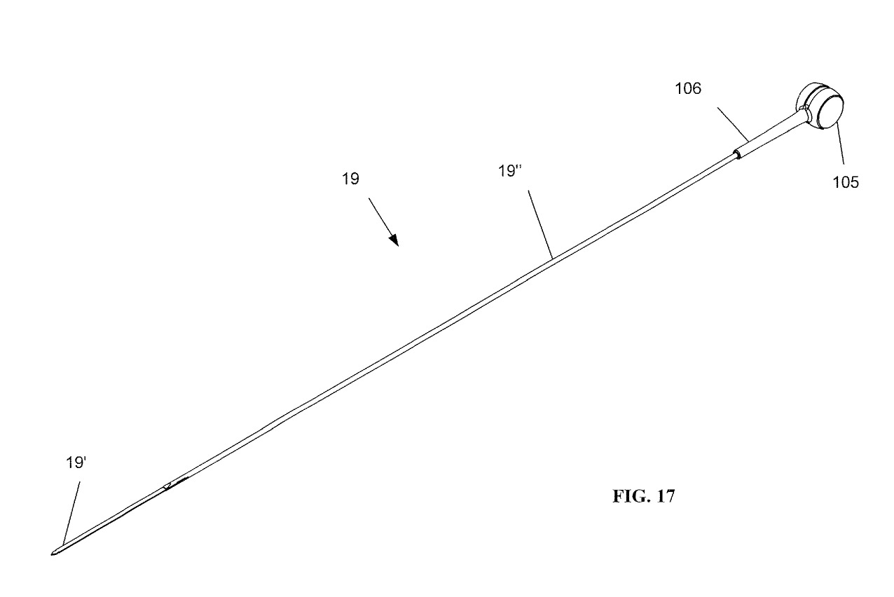

FIG. 17 is a perspective view of the suture needle apparatus 19. The suture needle apparatus 19 includes a suture needle tip 19′ and a suture needle body 19″. A protrusion 106 may extend outwardly from the suture needle body 19″. The protrusion 106 may be a cylinder attached to the suture needle body 19″. A circular member 105 is attached to an end of the suture needle body 19″. The circular member 105 is configured to slide within a circular guide 12′ (FIG. 3).

The exemplary suture passing device allows surgeons to control the suture passing process with a single hand while allowing the surgeon to use the other hand to control the arthroscope for visualization, thus facilitating the suture passing process and resulting in a shorter surgery time. A shorter surgery time reduces the amount of time a patient is under anesthesia and reduces the cost of the surgery to both the patient and medical facility.

The various aspects of this disclosure are provided to enable one of ordinary skill in the art to practice the present invention. Modifications to various aspects of a suture passer device and a suture needle presented throughout this disclosure will be readily apparent to those skilled in the art, and the concepts disclosed herein may be extended to other applications to interface with other digital audio players. Thus, the claims are not intended to be limited to the various aspects of the suture passer device and the suture needle presented throughout this disclosure, but are to be accorded the full scope consistent with the language of the claims. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

What is claimed is:

1. A suture passer device, comprising:
a shaft having a shaft proximal end and a shaft distal end;
a handle assembly coupled to the shaft proximal end;
an end effector coupled to the shaft distal end; and
a suture capturing member within the end effector, coupled to the handle assembly independently of the shaft, and configured to capture a suture within the end effector.

2. The suture passer device of claim 1, wherein the suture capturing member comprises a bar extending from the end effector to the handle assembly through the shaft and configured to move responsive to movement of the handle assembly.

3. The suture passer device of claim 1, wherein the suture capturing member is configured to move independently of the end effector longitudinally within the end effector.

4. The suture passer device of claim 1, wherein:
the handle assembly is configured to extend and to retract a suture needle apparatus; and
the suture capturing member is configured to move upon the suture needle apparatus exerting a force, within the handle assembly, on the suture capturing member.

5. The suture passer device of claim 1, wherein the end effector comprises a lower jaw and an upper jaw with an upper jaw opening.

6. The suture passer device of claim 5, wherein the handle assembly is configured to extend a suture needle apparatus through the lower jaw into the upper jaw opening and to retract the suture needle apparatus from the upper jaw opening into the lower jaw.

7. The suture passer device of claim 5, wherein the suture capturing member is configured to capture a suture between the suture capturing member and an inside edge of the upper jaw opening.

8. The suture passer device of claim 7, wherein the suture capturing member has a suture capturing member opening, and the suture capturing member is configured to capture a suture inserted into the upper jaw opening and the suture capturing member opening by pinching the suture between an inside edge of the suture capturing member opening and the inside edge of the upper jaw opening.

9. The suture passer device of claim 1, wherein the handle assembly comprises a handle, a hammer coupled to the suture capturing member, and a plurality of springs biasing the hammer between a first hammer position and a second hammer position.

10. The suture passer device of claim 9, wherein the handle assembly further comprises a cocking lever coupled to the hammer and configured to move the hammer from the first hammer position to the second hammer position, a locking mechanism configured to lock the hammer in the second hammer position, and a release button configured to release the locking mechanism from locking the hammer in the second hammer position.

11. The suture passer device of claim 9, wherein when the hammer is in the second hammer position, the suture capturing member is positioned to allow a suture to be inserted through the end effector; and when the hammer is in the first hammer position, the suture capturing member is positioned to capture the inserted suture within the end effector.

12. The suture passer device of claim 9, wherein the plurality of springs comprise a first spring and a second spring, the first spring biases the hammer toward the second hammer position and the second spring biases the hammer toward the first hammer position.

13. The suture passer device of claim 12, wherein the first spring has a higher rate than the second spring.

14. The suture passer device of claim 12, wherein:
the end effector comprises a lower jaw and an upper jaw with an upper jaw opening; and
the handle is configured to extend a suture needle apparatus comprising a suture needle through the lower jaw into the upper jaw opening, to retract the suture needle from the upper jaw opening into the lower jaw, and to apply a force via the suture needle apparatus on the first spring; and
the first spring and the second spring are configured to move the hammer into the second hammer position before the suture needle extends into the upper jaw opening and to delay the hammer from moving into the first hammer position until the suture needle is retracted from the upper jaw opening.

15. A suture passer device, comprising:
a shaft having a shaft proximal end and a shaft distal end;
a handle assembly coupled to the shaft proximal end;
an end effector coupled to the shaft distal end; and
means for capturing a suture within the end effector and configured to move responsive to a force applied within the handle assembly.

16. The suture passer device of claim 15, wherein the handle assembly comprises a first member coupled to the means for capturing the suture and the means for capturing the suture is configured to move responsive to the first member.

17. The suture passer device of claim 16, wherein:
the handle assembly is configured to extend and to retract a suture needle apparatus; and
the means for capturing a suture is configured to move upon the suture needle apparatus exerting said force on the first member.

18. The suture passer device of claim 17, wherein:
the handle assembly comprises a plurality of springs;
the first member is a hammer configured to move between a first hammer position and a second hammer position; and
the hammer is biased by the plurality of springs between the first hammer position and the second hammer position.

19. A suture needle apparatus, comprising:
a suture needle comprising a suture needle body and a suture needle tip;
a protrusion extending from the suture needle body; and
a circular member attached to an end of the suture needle body, the circular member being configured to slide within a circular guide.

20. The suture needle apparatus of claim 19, wherein the protrusion is a cylinder attached to the suture needle body.
